(19) United States
(12) Patent Application Publication
(10) Pub. No.: US 2009/0193897 A1
(43) Pub. Date: Aug. 6, 2009

(54) COMPOSITION FOR MATERIAL SENSING AND RELATED METHOD AND APPARATUS

(75) Inventors: Bogdan-Catalin Serban, Bucharest (RO); Sarinkumar A. Koyilothu, Bangalore (IN); Stefan-Dan Costea, Bucharest (RO); Cornel Cobianu, Bucharest (RO)

Correspondence Address: HONEYWELL INTERNATIONAL INC.;PATENT SERVICES, 101 COLUMBIA ROAD, P O BOX 2245, MORRISTOWN, NJ, 07962-2245, US

(73) Assignee: Honeywell International Inc., Morristown, NJ (US)

(21) Appl. No.: 12/363,842

(22) Filed: Feb. 2, 2009

(30) Foreign Application Priority Data
Feb. 6, 2008 (RO) A/2008-00100

(51) Int. Cl. G01N 29/00; C12Q 1/00

(52) U.S. Cl. 73/584; 435/4

(57) ABSTRACT

A sensor includes a piezoelectric substrate and conductive elements formed in or over the substrate. The sensor also includes a sensing layer formed over the substrate. The sensing layer has one or more properties (such as a mass loading, an electrical property, or a visco-elastic property) that vary based on at least one measurand to be measured by the sensor (such as carbon dioxide). This may affect, for example, a propagation velocity of acoustic waves in the sensor and/or a resonant frequency of the sensor. The sensing layer includes a combination of polyaniline and carbonic anhydrase. The combination of polyaniline and carbonic anhydrase could be formed using an emeraldine base. For instance, an aniline can be dissolved in water to form a mixture, and hydrochloric acid and an oxidant can be added to the mixture. A chemical polymerization of the aniline in the mixture can be performed to form polyanilne.

### TECHNICAL FIELD

This disclosure relates generally to material sensors and more specifically to a composition for material sensing and related method and apparatus.

### BACKGROUND

The detection and measurement of various materials are important functions in a wide variety of industries. For example, carbon dioxide detection and measurement are often desired or required functions in fields such as demand-control ventilation, food industry processing and transportation, capnography, geological research, green chemistry, and agricultural chemistry. The detection and measurement of carbon dioxide are often performed using carbon dioxide sensors, which typically include carbon dioxide-sensitive coatings.

Various carbon dioxide-sensitive coatings have been developed for use in these types of sensors. For example, organic polymers that contain amino groups are often used in carbon dioxide sensors. Example polymers include Versamid 900, polyethyleneimine, BMBT (N,N bis-(p-methoxybenzylidene)-a a′-bi-p-toluidine), and THEED (tetrakis(hydroxyethyl)ethylenediamine). However, these coatings often suffer from sensitivity towards water molecules in the surrounding environment, as well as sensitivity towards volatile organic compounds such as acetone, acetaldehyde, and ethanol.

Other common small organic molecules used in carbon dioxide sensors include: 7,10 dioxa-3,4 diaza-1, 5,12,16 hexadecatetrol; benzylamine; tri-n-octylamine; dipropylamine; 1,8 diamino-p-menthane; diphenylacetylene; and N,N diethyl-p-phenylendiamine. However, these coatings often exhibit a small frequency shift during operation in carbon dioxide sensors (such as a frequency shift of between 10 Hz and 210 Hz). These coatings also often suffer from poor stability and reproducibility.

### SUMMARY

This disclosure provides a composition for material sensing and related method and apparatus.

In a first embodiment, a sensor includes a piezoelectric substrate and first and second conductive elements formed in or over the substrate. The sensor also includes a sensing layer formed over the substrate. The sensing layer has one or more properties that vary based on at least one measurand to be measured by the sensor. The sensing layer includes a combination of polyaniline and carbonic anhydrase.

In particular embodiments, the first and second conductive elements include interdigital transducers. Also, a guiding layer that is capable of transporting acoustic waves between the interdigital transducers is formed over the substrate and the interdigital transducers. Further, the sensing layer is formed over the guiding layer. The sensing layer could be formed over an area of the guiding layer that is between the interdigital transducers, and the one or more properties of the sensing layer may affect a propagation velocity of the acoustic waves between the interdigital transducers.

In other particular embodiments, the first and second conductive elements include interdigital transducers, and the sensing layer is formed over the substrate and between the interdigital transducers. The one or more properties of the sensing layer may affect a propagation velocity of acoustic waves through a surface of the substrate between the interdigital transducers.

In yet other particular embodiments, the first and second conductive elements include electrodes having conductive plates, where the conductive plates are separated by the substrate. Also, the sensing layer includes multiple sensing layers each formed over one of the conductive plates. The one or more properties of the sensing layers may affect a resonant frequency of the sensor.

In still other particular embodiments, the one or more properties of the sensing layer may include a mass loading, an electrical property, and/or a visco-elastic property. Also, the at least one measurand may include carbon dioxide.

In a second embodiment, a method includes receiving a combination of polyaniline and carbonic anhydrase. The method also includes forming a sensing layer of a sensor using the combination of polyaniline and carbonic anhydrase. The sensing layer has one or more properties that vary based on at least one measurand to be measured by the sensor.

In particular embodiments, receiving the combination of polyaniline and carbonic anhydrase includes forming the combination of polyaniline and carbonic anhydrase. Forming the combination of polyaniline and carbonic anhydrase may include forming an emeraldine base having polyaniline and carbonic anhydrase.

In other particular embodiments, forming the combination of polyaniline and carbonic anhydrase includes dissolving an aniline in water to form a mixture, adding hydrochloric acid to the mixture, adding an oxidant to the mixture, and performing chemical polymerization of the aniline in the mixture.

In yet other particular embodiments, forming the combination of polyaniline and carbonic anhydrase further includes removing precipitated polyaniline from the mixture, dedoping the precipitated polyaniline, drying the dedoped polyaniline to form a powder, and mixing the powdered polyaniline with carbonic anhydrase in dimethylformamide.

In still other particular embodiments, forming the combination of polyaniline and carbonic anhydrase further includes adding poly (sodium-p-styrene sulfonate) to the mixture during the chemical polymerization, dedoping synthesized polyaniline in the mixture, and adding carbonic anhydrase to the mixture.

In a third embodiment, a composition includes a combination of polyaniline and carbonic anhydrase. In particular embodiments, the combination of polyaniline and carbonic anhydrase is soluble in dimethylformamide and/or water.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which:

FIGS. 1A and 1B illustrate a first example material sensor according to this disclosure;

FIGS. 2A and 2B illustrate a second example material sensor according to this disclosure;

FIGS. 3A through 3C illustrate a third example material sensor according to this disclosure;

FIG. 4 illustrates an example system using one or more material sensors according to this disclosure; and

FIGS. 5A and 5B illustrate example methods for forming a sensing layer in a material sensor according to this disclosure.

### DETAILED DESCRIPTION

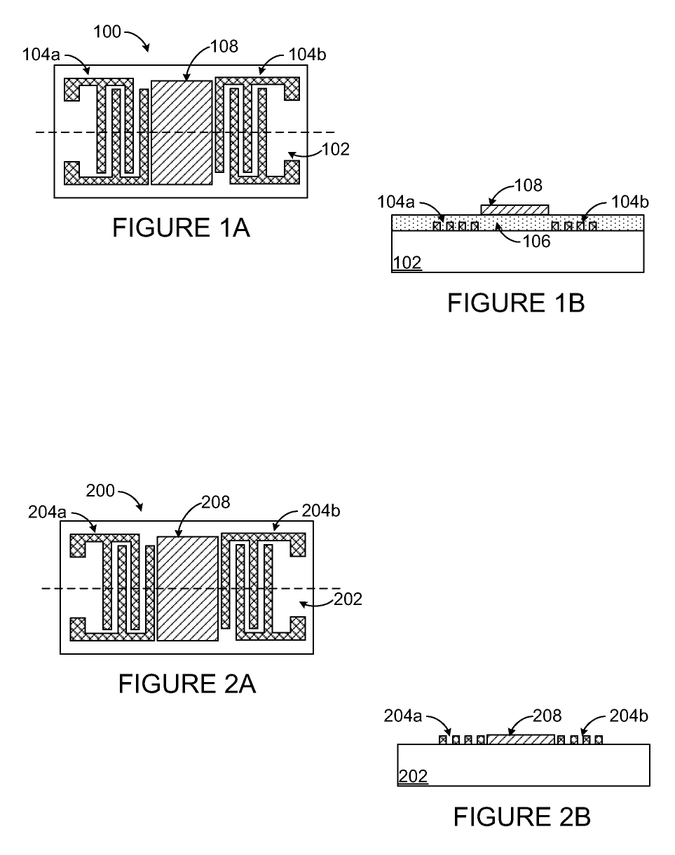

FIGS. 1A through 5, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

FIGS. 1A and 1B illustrate a first example material sensor 100 according to this disclosure. The embodiment of the sensor 100 shown in FIGS. 1A and 1B is for illustration only. Other embodiments of the sensor 100 could be used without departing from the scope of this disclosure.

In general, a material sensor is used to detect or measure one or more materials. In this document, the term “material” refers to any suitable substance being detected or measured. A material being detected or measured can be referred to as a “measurand.” Also, the detection or measurement of a material can take various forms depending on the implementation. For example, a sensor could be used to detect the presence of a material, such as when the sensor is used to detect whether a particular measurand is present at all or is present in at least a threshold amount or concentration. A sensor could also be used to measure a material, such as when the sensor is used to measure the amount or concentration of the material.

In this example, FIG. 1A illustrates the sensor 100, and FIG. 1B illustrates a cross-section of the sensor 100 (taken along the dashed line in FIG. 1A). As shown here, the sensor 100 includes a substrate 102. The substrate 102 generally represents any suitable piezoelectric structure on which other components of the sensor 100 are formed or carried. The substrate 102 could, for example, represent a substrate such as quartz.

In this example embodiment, the sensor 100 represents a surface acoustic wave (SAW) shared horizontal (SH) sensor formed using two interdigital transducers (IDTs) 104a-104b. As shown here, each of the interdigital transducers 104a-104b includes two sets of conductive fingers, where one set of conductive fingers is interleaved with the other set of conductive fingers. The interdigital transducer 104a could be viewed as the input transducer, and the interdigital transducer 104b could be viewed as the output transducer. As described in more detail below, during operation the interdigital transducer 104a produces acoustic waves based on an input signal. The waves propagate through the sensor 100 to the interdigital transducer 104b, which produces an output signal based on the waves. Each of the interdigital transducers 104a-104b could be formed using any suitable element(s) or compound(s) with high electrical conductivity, such as highly-doped polysilicon or metal. Each of the interdigital transducers 104a-104b could also be formed in any suitable manner, such as by etching the polysilicon or metal using a mask. In addition, each of the interdigital transducers 104a-104b could include any number of conductive fingers, depending (among other things) on the designed bandwidth and operational frequency of the interdigital transducers.

A guiding layer 106 is located over the substrate 102 and the interdigital transducers 104a-104b. The guiding layer 106 facilitates the transport of acoustic waves between the interdigital transducers 104a-104b. The guiding layer 106 could be formed using any suitable element(s) or compound(s), such as silicon dioxide or other dielectric. The guiding layer 106 could also be formed in any suitable manner, such as chemical vapor deposition, spin coating, and spray coating (where the preparation temperature of the guiding layer is below the Curie temperature of the piezoelectric substrate).

A sensing layer 108 is located over the guiding layer 106 (when viewed as shown in FIG. 1B) and between the interdigital transducers 104a-104b (when viewed as shown in FIG. 1A). The sensing layer 108 is generally exposed to the environment and is sensitive to one or more measurands being detected or measured. For example, the sensing layer 108 may be sensitive to carbon dioxide. The presence or level of the measurand typically alters one or more properties of the sensing layer 108. This affects the transport of the acoustic waves between the interdigital transducers 104a-104b, which can be determined by an external component (such as an external control circuit) and used to detect or measure the measurand. In this way, the sensing layer 108 provides a mechanism for identifying the presence or level of at least one measurand. Example compounds and techniques for forming the sensing layer 108 are provided below.

In particular embodiments, acoustic waves produced by exciting the input interdigital transducer 104a are guided through the guiding layer 106 to the output interdigital transducer 104b. The propagation velocity of the acoustic waves between the interdigital transducers 104a-104b may depend on the properties of both the guiding layer 106 and the sensing layer 108. At least one measurand (such as carbon dioxide) affects one or more properties of the sensing layer 108 (such as its mass loading, electrical, and visco-elastic properties). As a result, changes in the propagation velocity of the acoustic waves can be directly related to variations in the measurand. Propagation velocity changes in the sensor 100 can therefore be monitored and used to determine the presence or concentration of the measurand.

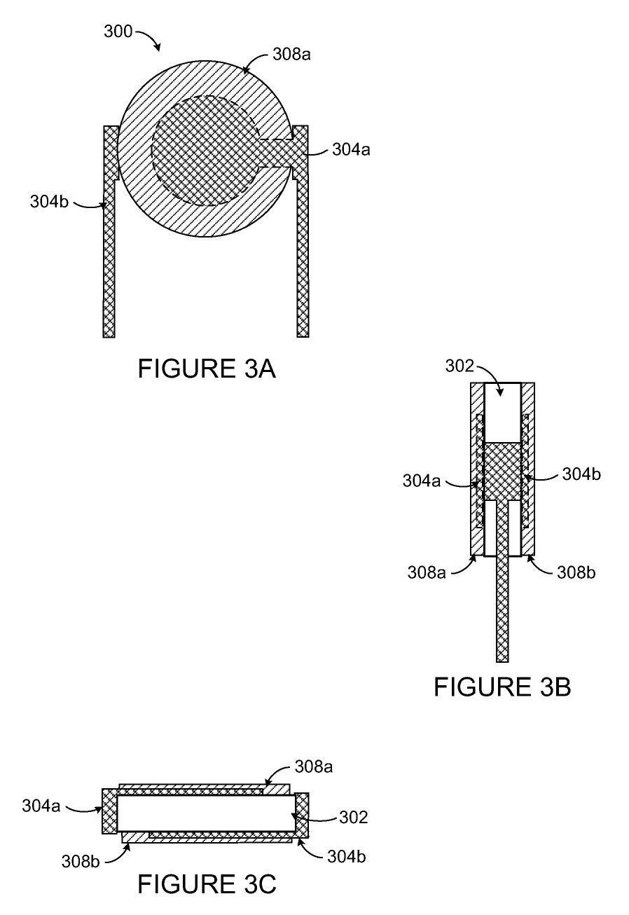

FIGS. 2A and 2B illustrate a second example material sensor 200 according to this disclosure. The embodiment of the sensor 200 shown in FIGS. 2A and 2B is for illustration only. Other embodiments of the sensor 200 could be used without departing from the scope of this disclosure.

In this example embodiment, the sensor 200 represents a SAW Rayleigh sensor that includes many of the same components as the sensor 100. For example, the sensor 200 includes a substrate 202, such as a quartz or other piezoelectric substrate. The sensor 200 also includes two interdigital transducers 204a-204b and a sensing layer 208. However, the sensor 200 lacks a guiding layer in this embodiment, and the sensing layer 208 is formed on the substrate 202.

In particular embodiments, acoustic waves produced by exciting the input interdigital transducer 204a can propagate through the surface of the substrate 202 to the output interdigital transducer 204b. The propagation velocity of the acoustic waves (which may also be guided through the sensing layer 208) may depend on the sensing layer's properties. At least one measurand affects one or more properties of the sensing layer 208 (such as its mass loading, electrical, and visco-elastic properties). As a result, changes in the propagation velocity of the acoustic waves can be directly related to variations in the measurand, and propagation velocity changes in the sensor 200 can be monitored and used to determine the presence or concentration of the measurand.

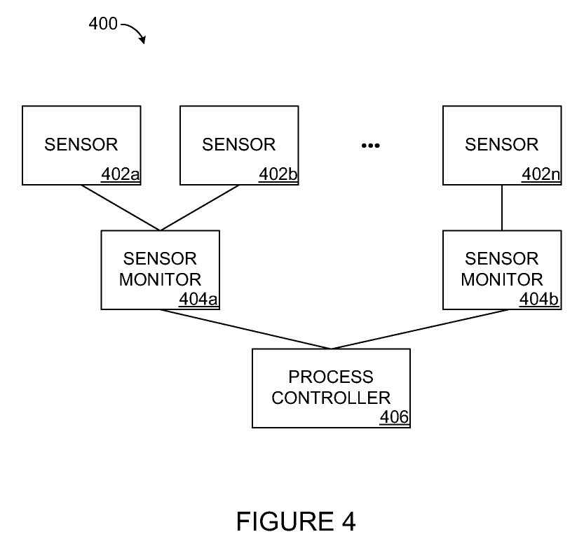

FIGS. 3A through 3C illustrate a third example material sensor 300 according to this disclosure. The embodiment of the sensor 300 shown in FIGS. 3A through 3C is for illustration only. Other embodiments of the sensor 300 could be used without departing from the scope of this disclosure.

In this example, the sensor 300 represents a bulk acoustic wave (BAW) sensor having a substrate 302, two electrodes 304a-304b, and two sensing layers 308a-308b. The substrate 302 represents any suitable substrate, such as a piezoelectric substrate like quartz. The electrodes 304a-304b include conductive plates that are separated from each other by the substrate 302. The electrodes 304a-304b could be formed in any suitable manner using any suitable element(s) or compound(s), such as metal. The conductive plates of the electrodes 304a-304b could also have any suitable shape and need not be circular. The conductive plates of the electrodes 304a-304b are covered by the sensing layers 308a-308b.

In particular embodiments, a radio frequency (RF) or other time-varying electrical signal is applied between the electrodes 304a-304b, producing bulk acoustic waves through the piezoelectric substrate 302. The resonant frequency at which the energy is absorbed by the substrate 302 may have a maximum value, which can depend not only on the characteristics of the piezoelectric substrate (such as thickness and cut) but also on the properties of the sensing layers 308a-308b. When the sensor 300 is exposed to at least one measurand, the measurand changes one or more properties of the sensing layers 308a-308b, which affects the resonant frequency of the sensor 300. Changes in the resonant frequency of the sensor 300 can be monitored and used to determine the presence or concentration of the measurand.

In accordance with this disclosure, the sensing layers used in the sensors 100-300 shown in FIGS. 1A through 3C (or other sensors) may be formed using a combination of polyaniline and carbonic anhydrase. Sensing layers implemented in this way may be used, for example, to detect or measure carbon dioxide molecules at room temperatures in SAW or BAW sensors. These types of sensors may operate under low or high humidity atmospheres and may have higher sensitivities compared to conventional sensors.

In some embodiments, the polyaniline used to form the sensing layers may be unsubstituted or substituted and may be soluble in organic solvents or water. The polyaniline could, for example, be used in the form of an emeraldine base that acts as an insulator. The polyaniline possesses nitrogen atoms, which are susceptible to being protonated with protons that come from the reaction between carbon dioxide and water. The carbonic anhydrase acts as a catalyst for the conversion of carbon dioxide to bicarbonate and protons.

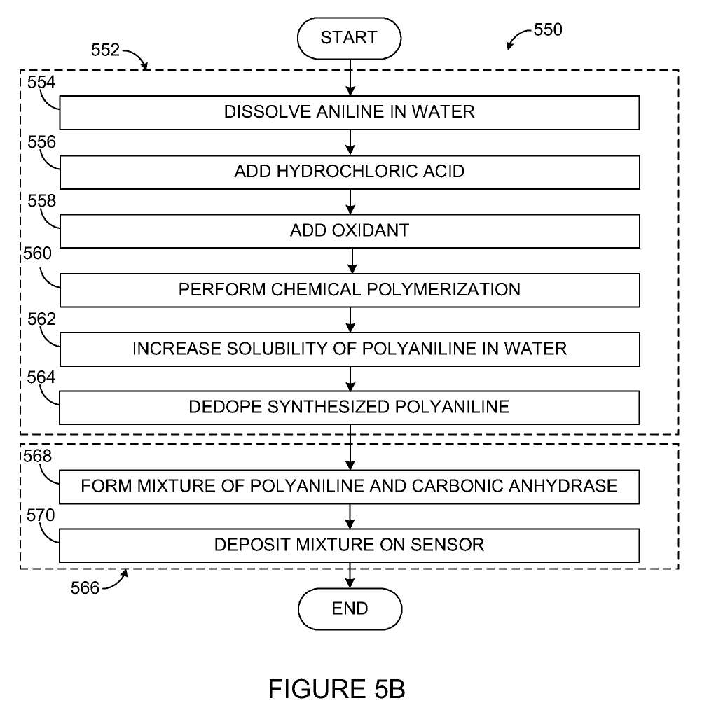

This reaction can be used to detect the presence of or to measure the concentration of carbon dioxide. For example, during protonation of nitrogen atoms, the conductivity of the sensing layer increases. In the sensor 100 of FIG. 1, this causes acoustic waves to extend into the sensing layer 108, and the propagation velocity of the waves depend on the physical characteristics of the sensing layer 108. Similarly, in FIG. 2, a change in the conductivity of the sensing layer 208 changes the electric field created by the acoustic waves, thereby changing the waves' propagation velocity. This is the so-called acousto-electric interaction between the sensing layer and the piezoelectric substrate, which is explained by the piezoelectric effect between propagating acoustic waves and the changing electric field in the sensing layer. These changes can be measured and used to determine the presence or concentration of one or more measurands. For instance, the propagation velocity can be monitored by recording an electrical signal time delay from the input to the output of the sensor. In sensors such as the sensor 300 of FIG. 3, the resonant frequency is directly related to the propagation velocity, and variations in the resonant frequency can be used to extract information about the measurand. Example techniques for forming the sensing layers are provided below in FIGS. 5A and 5B.

Although FIGS. 1A through 3C illustrate three example material sensors, various changes may be made to these sensors. For example, the sensors shown and described above are for illustration only. One or more sensing layers formed using a combination of polyaniline and carbonic anhydrase could be used with any other suitable sensor or other device. Also, the layout and arrangement of components in the sensors shown in these figures could be altered according to particular needs.

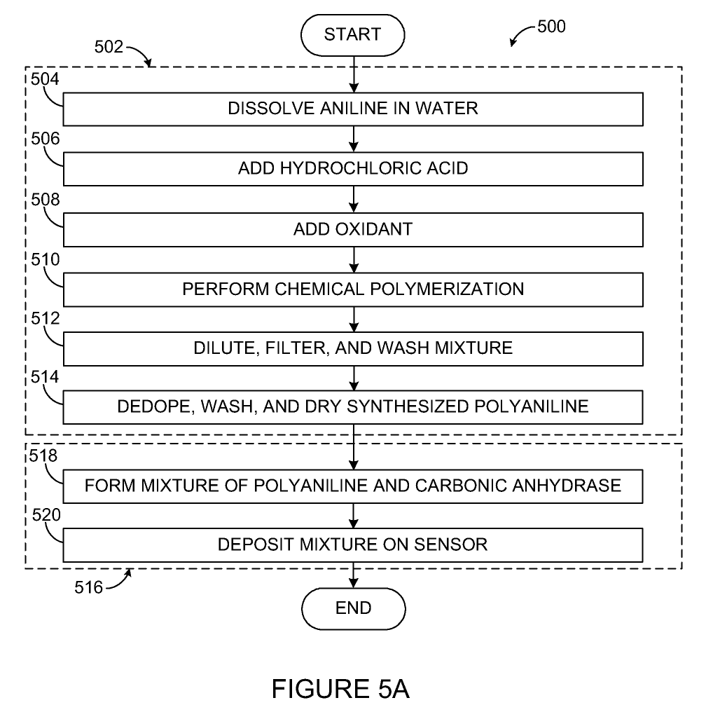

FIG. 4 illustrates an example system 400 using one or more material sensors according to this disclosure. The embodiment of the system 400 shown in FIG. 4 is for illustration only. Other embodiments of the system 400 could be used without departing from the scope of this disclosure.

In this example, the system 400 includes sensors 402a-402n, sensor monitors 404a-404b, and a process controller 406. The sensors 402a-402n can be distributed in one or more areas being monitored, such as in different portions of an industrial facility, geological structure, or other area. Each of the sensors 402a-402n includes at least one sensing layer formed using a combination of polyaniline and carbonic anhydrase. Each of the sensors 402a-402n could, for example, represent one of the sensors 100-300 described above. In this document, the interdigital transducers, electrodes, and other conductive structures in a sensor may be collectively referred to as “conductive elements.”

Each of the sensors 402a-402n is coupled to one or more of the sensor monitors 404a-404b. The sensor monitors 404a-404b use the sensors 402-402n to detect or measure one or more measurands. For example, each sensor 402a-402n could be located in the feedback loop of an oscillator included in or associated with one of the sensor monitors 404a-404b, and such an oscillator can change its operational frequency as a function of the material to be measured. The sensor monitors 404a-404b could use signals from the sensors 402a-402n to detect or measure one or more measurands in the environment near the sensors. For instance, the sensor monitor 404a-404b could use the signals to identify changes in the propagation velocity of acoustic waves in the sensor or to identify changes in the resonant frequency of the sensor. The sensor monitor 404a-404b could then use these changes to identify, for example, the presence, amount, or concentration of one or more measurands that are causing the changes. The sensor monitor 404a-404b could process this information further (such as by determining if a threshold has been exceeded and triggering an output if so) , or the sensor monitor 404a-404b could output the measurement data to the process controller 406. Each of the sensor monitors 404a-404b includes any suitable structure for using signals from one or more sensors to detect or measure one or more measurands.

The process controller 406 controls at least one process (or portion thereof) based on outputs of the sensor monitors 404a-404b. For example, the process controller 406 could receive an indication from one of the sensor monitors 404a-404b that a concentration or amount of a measurand has been detected or has exceeded a threshold. The process controller 406 could also receive measurement data from the sensor monitors 404a-404b and determine itself that a concentration or amount of a measurand has been detected or has exceeded a threshold. The process controller 406 could then take any suitable action. For instance, if the measurand is carbon dioxide, the process controller 406 could trigger an alarm or initiate venting of a particular area. The process controller 406 includes any hardware, software, firmware, or combination thereof for controlling at least one process or portion thereof based on sensor data from one or more sensors 402a-402n.

Each of the connections between components in FIG. 4 could represent any suitable wired or wireless connection. For example, the sensors 402a-402n could be physically wired to the sensor monitors 404a-404b, and the sensor monitors 404a-404b could be in wireless communication with the process controller 406.

Although FIG. 4 illustrates one example of a system 400 using one or more material sensors, various changes may be made to FIG. 4. For example, each sensor may be coupled to any number of monitors, and each monitor could be coupled to any number of sensors. Also, the functional division shown in FIG. 4 is for illustration only. Various components in FIG. 4 could be combined, subdivided, or omitted and additional components could be added according to particular needs. As a specific example, some or all of the functionality of the sensor monitors could be incorporated into the process controller or vice versa.

FIGS. 5A and 5B illustrate example methods for forming a sensing layer in a material sensor according to this disclosure. The embodiments of the methods shown in FIGS. 5A and 5B are for illustration only. Other embodiments of the methods could be used without departing from the scope of this disclosure.

In FIG. 5A, a sensing layer for carbon dioxide or other sensors is formed using a combination of polyaniline and carbonic anhydrase. In this example embodiment, the polyaniline is soluble in organic solvents such as dimethylformamide and N-methyl pirrolidone.

A method 500 in FIG. 5A is divided into two main steps. During the first main step 502, a polyaniline is formed as an emeraldine base. In this example, an aniline (such as 30 g of 2-methoxy aniline) is dissolved in water (such as 270 mL of water) at step 504. Hydrochloric acid (such as 50 mL of 10N hydrochloric acid) is slowly added to the aniline-water mixture at step 506. During or after this step, the reaction mixture can be stirred in an ice bath, such as by using a mechanical stirring tool for one hour. An oxidant (such as 60 g of ammonium peroxodisulphate) is added to the mixture at step 508. At this point, chemical polymerization of the aniline can occur at step 510. During the polymerization, the mixture could be refrigerated (such as at 4° C.) for a suitable length of time (such as for five hours) to provide sufficient time for the completion of the chemical polymerization. The mixture is then diluted, filtered, and washed at step 512. For example, the reaction mixture can be diluted with water, precipitated polyaniline can be filtered under vacuum, and the crude polyaniline can be washed with distilled water (in two portions). The synthesized polyaniline is then dedoped, washed, and dried at step 514. This may include adding an excess of 2M ammonium hydroxide to the crude polyaniline-water mixture in order to perform the dedoping of the synthesized polyaniline. This may also include washing the mixture with distilled water and drying the resulting dedoped poly (2-methoxy) aniline (such as at 80° C. for three hours) to form powdered poly (2-methoxy) aniline.

The second main step 516 of the method 500 shown in FIG. 5A is the application of at least one sensing layer to a sensor. This can include forming a mixture of polyaniline and carbonic anhydrase at step 518. For example, the powdered poly (2-methoxy) aniline formed during step 514 (such as 2 g) and carbonic anhydrase (such as 0.1 g) can be dissolved in a freshly distilled dimethylformamide. This can form a homogeneous solution, which is deposited onto the surface of one or more sensors at step 520. The deposition could occur in any suitable manner, such as using a drop casting technique.

In FIG. 5B, a sensing layer for carbon dioxide or other sensors is formed using a combination of polyaniline-poly (sodium p-styrene sulfonate)-carbonic anhydrase, which is soluble in water. In this example embodiment, the monomer is a simple aniline or substituted aniline in ortho or meta positions, and the substituents may be methoxy, ethoxy, or propoxy groups.

Again, the method 550 in FIG. 5B is divided into two main steps. During the first main step 552, a polyaniline is formed as an emeraldine base. In this example, an aniline (such as 30 g of 2-methoxy aniline) is dissolved in water (such as 270 mL of water) at step 554. Hydrochloric acid (such as 50 mL of 10N hydrochloric acid) is slowly added to the aniline-water mixture at step 556. This reaction mixture can be stirred in an ice bath, such as by using a mechanical stirring tool for one hour. An oxidant (such as 60 g of ammonium peroxodisulphate) is added to the mixture at step 558. At this point, chemical polymerization of the aniline can occur at step 560. During the polymerization, the solubility of the polyaniline in water is increased at step 562, such as by adding a small quantity of poly(sodium-p-styrene sulfonate). After that, the mixture could be refrigerated (such as at 4° C.) for a suitable length of time (such as for five hours) to provide sufficient time for the completion of the chemical polymerization. The synthesized polyaniline is then dedoped at step 564, such as by adding an excess of 2M ammonium hydroxide to the mixture.

The second main step 566 of the method 550 shown in FIG. 5B is the application of at least one sensing layer to a sensor. This can include forming a mixture of polyaniline and carbonic anhydrase at step 568. For example, carbonic anhydrase (such as 20 mg) can be added to the synthesized polyaniline mixture produced at step 564. This can form a homogeneous solution, which is deposited onto the surface of one or more sensors at step 570. The deposition could occur in any suitable manner, such as by using a spin coating technique.

Although FIGS. 5A and 5B illustrates example methods for forming a sensing layer in a material sensor, various changes may be made to FIGS. 5A and 5B. For example, while shown as forming a polyaniline as an emeraldine base, polyanilines in any other form could be used here. Also, any other suitable mixture of polyaniline and carbonic anhydrase could be used in these methods.

It may be advantageous to set forth definitions of certain words and phrases used throughout this patent document. The term “couple” and its derivatives refer to any direct or indirect communication between two or more elements, whether or not those elements are in physical contact with one another. The terms “over” and “above” denote relative positions of two layers or other elements in a particular orientation and do not require direct contact between the two layers or other elements. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The term “controller” means any device, system, or part thereof that controls at least one operation. A controller may be implemented in hardware, firmware, software, or some combination of at least two of the same. The functionality associated with any particular controller may be centralized or distributed, whether locally or remotely.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A sensor comprising:
a piezoelectric substrate;
a first conductive element and a second conductive element formed in or over the substrate; and
a sensing layer formed over the substrate, the sensing layer having one or more properties that vary based on at least one measurand to be measured by the sensor, the sensing layer comprising a combination of polyaniline and carbonic anhydrase.

2. The sensor of claim 1, wherein:
the first and second conductive elements comprise interdigital transducers;
a guiding layer capable of transporting acoustic waves between the interdigital transducers is formed over the substrate and the interdigital transducers; and
the sensing layer is formed over the guiding layer.

3. The sensor of claim 2, wherein:
the sensing layer is formed over an area of the guiding layer that is between the interdigital transducers; and
the one or more properties of the sensing layer affect a propagation velocity of the acoustic waves between the interdigital transducers.

4. The sensor of claim 1, wherein:
the first and second conductive elements comprise interdigital transducers; and
the sensing layer is formed over the substrate and between the interdigital transducers.

5. The sensor of claim 4, wherein the one or more properties of the sensing layer affect a propagation velocity of acoustic waves through a surface of the substrate between the interdigital transducers.

6. The sensor of claim 1, wherein:
the first and second conductive elements comprise electrodes having conductive plates, the conductive plates separated by the substrate; and
the sensing layer comprises multiple sensing layers each formed over one of the conductive plates.

7. The sensor of claim 6, wherein the one or more properties of the sensing layers affect a resonant frequency of the sensor.

8. The sensor of claim 1, wherein the one or more properties of the sensing layer comprise at least one of: a mass loading, an electrical property, and a visco-elastic property.

9. The sensor of claim 1, wherein the at least one measurand comprises carbon dioxide.

10. A method comprising:
receiving a combination of polyaniline and carbonic anhydrase; and
forming a sensing layer of a sensor using the combination of polyaniline and carbonic anhydrase, the sensing layer having one or more properties that vary based on at least one measurand to be measured by the sensor.

11. The method of claim 10, wherein receiving the combination of polyaniline and carbonic anhydrase comprises forming the combination of polyaniline and carbonic anhydrase.

12. The method of claim 11, wherein forming the combination of polyaniline and carbonic anhydrase comprises forming an emeraldine base comprising polyaniline and carbonic anhydrase.

13. The method of claim 11, wherein forming the combination of polyaniline and carbonic anhydrase comprises:
dissolving an aniline in water to form a mixture;
adding hydrochloric acid to the mixture;
adding an oxidant to the mixture; and
performing chemical polymerization of the aniline in the mixture.

14. The method of claim 13, wherein forming the combination of polyaniline and carbonic anhydrase further comprises:
removing precipitated polyaniline from the mixture;
dedoping the precipitated polyaniline;
drying the dedoped polyaniline to form a powder; and
mixing the powdered polyaniline with carbonic anhydrase in dimethylformamide.

15. The method of claim 13, wherein forming the combination of polyaniline and carbonic anhydrase further comprises:
adding poly(sodium-p-styrene sulfonate) to the mixture during the chemical polymerization;
dedoping synthesized polyaniline in the mixture; and
adding carbonic anhydrase to the mixture.

16. The method of claim 10, wherein the one or more properties of the sensing layer affect at least one of: a propagation velocity of acoustic waves in the sensor and a resonant frequency of the sensor.

17. The method of claim 10, wherein the one or more properties of the sensing layer comprise at least one of: a mass loading, an electrical property, and a visco-elastic property.

18. The method of claim 10, wherein the at least one measurand comprises carbon dioxide.

19. A composition comprising a combination of polyaniline and carbonic anhydrase.

20. The composition of claim 19, wherein the combination of polyaniline and carbonic anhydrase is soluble in at least one of: dimethylformamide and water.
